(19) United States
(12) Patent Application Publication
(10) Pub. No.: US 2012/0232031 A1
(43) Pub. Date: Sep. 13, 2012

(54) INJECTABLE FORMULATIONS FOR INTRA-ARTICULAR OR PERI-ARTICULAR ADMINISTRATION

(75) Inventors: John Howard Hutchinson, San Diego, CA (US); Petpiboon Peppi Prasit, Rancho Santa Fe, CA (US); Isabelle Marguerite Dearmond, San Diego, CA (US)

(73) Assignee: Panmira Pharmaceuticals, LLC, San Diego, CA (US)

(21) Appl. No.: 13/502,488

(22) PCT Filed: Oct. 19, 2010
(86) PCT No.: PCT/US10/53245

§ 371 Date: May 18, 2012

Related U.S. Application Data
(60) Provisional application No. 61/252,975, filed on Oct. 19, 2009.

(51) Int. Cl. A61K 31/404; A61K 31/4709; A61K 31/47; A61K 31/506; A61K 31/444; A61K 31/381; A61K 31/4245; A61K 31/4178; A61K 31/662; A61K 31/222; A61K 31/4745; A61K 31/196; A61K 31/265; A61K 31/4045; A61K 31/427; A61K 31/726; A61K 31/7008; A61P 19/02; A61P 29/00; A61P 31/04; A61P 31/10; A61P 37/08; A61P 31/12; A61P 9/00; A61P 35/00; A61P 23/00; A61K 31/405

(52) U.S. Cl. 514/54; 514/418; 514/314; 514/311; 514/269; 514/255.05; 514/333; 514/443; 514/438; 514/312; 514/364; 514/397; 514/134; 514/546; 514/279; 514/563; 514/512; 514/415; 514/369; 514/62

(57) ABSTRACT

Described herein are injectable formulations for intra-articular or peri-articular administration, wherein the formulation is administered to treat joint pain. An injectable formulation for intra-articular or peri-articular administration disclosed herein comprises a therapeutically-effective amount of a leukotriene synthesis inhibitor compound formulated for intra-articular or peri-articular administration.

### RELATED APPLICATIONS

This application claims the benefit of U.S provisional patent application No. 61/252,975 entitled “INJECTABLE FORMULATIONS FOR INTRA-ARTICULAR OR PERI-ARTICULAR ADMINISTRATION” filed on Oct. 19, 2009, which is incorporated by reference in its entirety.

### FIELD OF THE INVENTION

Described herein are injectable formulations for intra-articular or peri-articular administration to a mammal that include a compound that inhibits synthesis of leukotrienes and other 5-lipoxygenase products and methods of use thereof in the treatment or prevention of joint pain.

### BACKGROUND OF THE INVENTION

Joint pain stems from abnormal states, conditions, disorders, or disease of a joint and/or a related tissue. By way of non-limiting example, abnormal states, conditions, disorders, or disease of a joint and/or a related tissue include inflammatory joint disease, ankylosis, synovitis, arthralgias, rheumatism, spondylitis, bursitis, brucellosis, gout, chondrocalcinosis, degenerative joint disease, lupus erythematosus, arthritis, osteoarthritis, infectious arthritis, Still's disease, onchronotic arthropathy, mucopolysaccharidoses, osteochondritis dissecans, synovial chondromatosis, osteochondromatosis, synoviomas, diabetes, Lyme disease, dysplasia, joint injury, sprains, dislocations, tears in ligaments and tendons, hemarthrosis, scarring or following joint surgery or combinations thereof.

### SUMMARY OF THE INVENTION

Described herein, in certain embodiments, are injectable formulations for intra-articular or peri-articular administration, wherein the formulation is administered to treat joint pain or inflammation. Described herein, in certain embodiments, are injectable formulations for administration into joints. The injectable formulation is formulated with excipients that are acceptable for intra-articular or peri-articular administration.

In certain embodiments, the formulation is administered to treat joint pain or inflammation associated an immune disorder (e.g. an autoimmune disorder); a proliferation disorder (e.g., synoviomas); contact with an allergen, and/or an irritant; a fibroblast disorder (e.g., scarring); or combinations thereof.

Described herein, in certain embodiments, are injectable formulations for intra-articular or peri-articular administration, wherein the formulation is administered to treat pain associated with inflammatory joint disease, ankylosis, synovitis, arthraglias, rheumatism, spondylitis, bursitis, brucellosis, gout, chondrocalcinosis, degenerative joint disease, lupus erythematosus, arthritis, osteoarthritis, infectious arthritis, Still's disease, onchronotic arthropathy, mucopolysaccharidoses, osteochondritis dissecans, synovial chondromatosis, synoviomas, diabetes, Lyme disease, dysplasia, joint injury, sprains, dislocations, tears in ligaments and tendons, hemarthrosis, scarring or following joint surgery or combinations thereof.

In some embodiments, an injectable formulation for intra-articular or peri-articular administration disclosed herein comprises a therapeutically-effective amount of a 5-lipoxygenase-activating protein (FLAP), a 5-LO inhibitor, or a cPLA₂ inhibitor, or a pharmaceutically acceptable salt thereof.

In some embodiments, described is an injectable formulation comprising a leukotriene synthesis inhibitor compound in an amount effective for the treatment of joint pain or inflammation in a mammal, and at least one suitable pharmaceutically acceptable excipient for intra-articular or peri-articular administration.

In some embodiments, described is an injectable formulation comprising a leukotriene synthesis inhibitor compound in an amount effective for inhibiting leukotriene synthesis in a joint of a mammal, and at least one suitable pharmaceutically acceptable excipient for intra-articular or peri-articular administration.

In some embodiments, described is an injectable formulation comprising a leukotriene synthesis inhibitor in an amount effective for antagonizing a leukotriene receptor, and at least one suitable pharmaceutically acceptable excipient for intra-articular or peri-articular administration.

In some embodiments, described is an injectable formulation comprising a leukotriene synthesis inhibitor in an amount effective for reducing or inhibiting the pain in a joint of a mammal, and at least one suitable pharmaceutically acceptable excipient for intra-articular or peri-articular administration.

In some embodiments, described is a method of treating of joint pain in a mammal comprising intra-articularly or peri-articularly administering an injectable formulation comprising a therapeutically-effective amount of a leukotriene synthesis inhibitor compound to at least one joint of a mammal with a joint disease, disorder or condition.

In some embodiments, described is a method of inhibiting leukotriene synthesis in a joint of a mammal comprising administering an intra-articular or peri-articular injectable formulation comprising a leukotriene synthesis inhibitor compound to a joint of the mammal.

In some embodiments, the leukotriene synthesis inhibitor is an inhibitor of 5-lipoxygenase activating protein (FLAP). In some embodiments, the leukotriene synthesis inhibitor is an inhibitor of 5-lipoxygenase (5-LO). In some embodiments, the leukotriene synthesis inhibitor is an inhibitor of cytosolic PLA₂ (c-PLA₂).

In some embodiments, described is a method of reducing the amount of or inhibiting pain in a joint of a mammal comprising administering an intra-articular or peri-articular injectable formulation comprising a leukotriene synthesis inhibitor compound to a joint of the mammal.

In some embodiments, an injectable formulation for intra-articular or peri-articular administration disclosed herein is administered before or after contact with an allergen and/or irritant and/or an infectious agent (e.g., a virus). In some embodiments, an injectable formulation for intra-articular or peri-articular administration disclosed herein is administered before or after a physical trauma (e.g., surgery, or injury). In some embodiments, the injectable formulations described herein are administered for conditions associated with the articular capsule surrounding the joint. In other embodiments, the injectable formulations are intra-articular administered to the joint, but subsequently penetrate into the related tissues to treat a disease associated joint pain or inflammation. In other embodiments, the injectable formulations are administered in the synovial cavity of the joint and related tissues: non-limiting examples include the synovial fluid, synovial membrane, articular cartilage and ligaments.

In some embodiments, compounds provided herein are administered to a human. In some embodiments, compounds provided herein are administered to a human as a pharmaceutically acceptable salt.

In some embodiments, provided is an article of manufacture, which includes packaging material, an injectable formulation within the packaging material, and a label that indicates that the injectable formulation is used for intra-articular or peri-articular administration.

Other objects, features and advantages of the topical formulations described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

5-Lipoxygenase products including leukotrienes are a class of pro-inflammatory lipid mediators derived from arachidonic acid that have been shown to play important roles in a number of biological processes. Arachidonic acid is released from the sn-2 position of glycerophospholipids through the action of the enzyme phospholipase A₂ (PLA₂). Phospholipases A₂ include several protein families with common enzyme activity and they include secreted PLA₂ (sPLA₂), cytosolic PLA₂ (cPLA₂), Ca²⁺ independent PLA₂ (iPLA₂) and lipoprotein-associated PLA₂ (lp-PLA₂). 5-Lipoxygenase products are mainly derived from arachidonic acid released by cPLA₂. Arachidonic acid is converted to leukotriene A₄ (LTA₄) in a two-step process, with 5-hydroperoxyeicosatetraenoic acid (5-HPETE) as an intermediate that can be converted to 5-hydroxyeicosatetraenoic acid (5-HETE) and 5-oxo-eicosatetraenoic acid (5-oxo-ETE), mediated by the enzyme 5-lipoxygenase (5-LO). LTA₄ is converted either to LTB₄ via LTA₄ hydrolase or to LTC₄ through conjugation with glutathione mediated by LTC₄ synthase. Serum dipeptidase carries out an amide bond cleavage to convert LTC₄ to LTD₄ and then gamma-glutamyl transpeptidase removes glutamate from LTD₄ to produce LTE₄. The initial oxidation step is a process that requires the intimate involvement of both 5-LO and the membrane-bound 5-lipoxygenase-activating protein (FLAP) Inhibition of FLAP or 5-LO results in the inhibition of all cellular bioactive leukotriene production and also inhibits the cellular production of 5-HETE and 5-oxo-ETE. LTB₄ is the ligand for the G protein-coupled receptors (GPCRs) BLT₁ and BLT₂ and both receptors are involved in chemotaxis and cell stimulation in the inflammatory response. It is contemplated that 5-oxo-ETE is the ligand for the G protein-coupled receptor OXE and activation of this receptor stimulates eosinophil and neutrophil chemotaxis and basophil histamine release.

Leukotrienes are lipid mediators of inflammation that are involved in the pathogenesis of joint pain and associated disorders. Leukotrienes are produced mainly by mast cells, eosinophils, monocytes/macrophages, and neutrophils, or by transcellular metabolism in platelets and endothelial cells, in response to allergic and/or inflammatory stimuli. Leukotrienes sensitize the pain fiber to be excitable and activated by a smaller stimulus. Light pressure that is not normally perceived to be painful, is sometimes sensed as pain when leukotrienes surround the pain fiber. In one aspect, biological tissues in areas that are affected by joint pain and the associated disorder have high concentrations of leukotrienes. The role of cPLA₂, FLAP and 5-LO in the leukotriene synthesis pathway is significant because cPLA₂ releases arachidonic acid that is used for leukotriene synthesis. FLAP in concert with 5-LO perform the first step in the pathway for the synthesis of leukotrienes. Inhibiting cPLA₂, FLAP and/or 5-LO provides a target for the treatment of leukotriene-dependent or leukotriene-mediated joint pain associated with joint diseases, disorders or conditions, including, by way of example, inflammatory joint disease, ankylosis, synovitis, arthralgias, rheumatism, spondylitis, bursitis, brucellosis, gout, chondrocalcinosis, degenerative joint disease, lupus erythematosus, arthritis, osteoarthritis, infectious arthritis, Still's disease, onchronotic arthropathy, mucopolysaccharidoses, osteochondritis dissecans, synovial chondromatosis, osteochondromatosis, synoviomas, diabetes, Lyme disease, dysplasia, joint injury, sprains, dislocations, tears in ligaments and tendons, hemarthrosis, scarring or following joint surgery or combinations thereof.

In one aspect, leukotrienes are involved in the pathogenesis of joint pain associated with diseases, disorders or conditions. Joint diseases, disorders or conditions are treated or prevented by intra-articular administration of a pharmaceutical composition that includes a cPLA₂ or FLAP or 5-LO inhibitor compound.

Disclosed herein is the use of cPLA₂, FLAP or 5-LO inhibitors in the manufacture of medicaments suitable for intra-articular administration to a joint of a mammal for the treatment or prevention of leukotriene-dependent or leukotriene-mediated or 5-lipoxygenase product-dependent or 5-lipoxygenase product-mediated joint pain.

As used herein, “leukotriene synthesis inhibitors” or “leukotriene synthesis inhibitor compounds” refer to compounds that inhibit the production of 5-lipoxygenase products through the inhibition of cPLA₂, FLAP and/or 5-LO. In some embodiments, the leukotriene synthesis inhibitor has a short systemic half life in the mammal. In some embodiment, a short systemic half life in the mammal is less than 6 hours, less than 5 hours, less than 4 hours, less than 3 hours, less than 2 hours, less than 1 hour, or less than 30 minutes.

Described herein are pharmaceutical formulations suitable for intra-articular or peri-articular administration, methods for treating, methods for formulating a injectable formulation for intra-articular administration, methods for producing, methods for manufacturing, treatment strategies, using a leukotriene synthesis inhibitor.

Described herein, in certain embodiments, are injectable formulations for intra-articular or peri-articular administration that include a leukotriene synthesis inhibitor compound, wherein the formulation is administered to treat joint pain or inflammation. In one aspect, intra-articular administration of a leukotriene synthesis inhibitor compound to a joint of a mammal minimizes systemic absorption of the leukotriene synthesis inhibitor compound. In one aspect, intra-articular administration of a leukotriene synthesis inhibitor compound to a joint provides for local treatment of joint pain or inflammation. In one aspect, local treatment of joint pain or inflammation with a leukotriene synthesis inhibitor compound reduces possible side effects associated with systemic administration of a leukotriene synthesis inhibitor compound.

In some embodiments, an intra-articular formulation described herein comprises a leukotriene synthesis inhibitor compound in combination with an antibiotic; anti-fungal agent; steroid anti-inflammatory agent; non-steroidal anti-inflammatory agent; antihistamine; antiviral; alpha agonist; beta blocker; carbonic anhydrase inhibitor; prostaglandin; anti-angiogenesis agent; mast cell stabilizer; glycosaminoglycan; anti-cancer agent; analgesics, lubricating agents, p38 kinase inhibitors, chemokine inhibitors, anti-TNF alpha agents, and/or anesthetics to treat or prevent a joint pain.

In some embodiments, an intra-articular formulation described herein comprises a leukotriene synthesis inhibitor compound administered prior to or following surgery to treat post-surgical pain. In one aspect, the joint pain is a result of the over-production of leukotrienes or other 5-lipoxygenase products.

In one aspect, the joint pain is associated with, but is not limited to, joint inflammatory disorders, joint immune disorders, joint proliferative disorders, a joint disorder resulting from contact with an allergen and/or an irritant, an joint fibroblast disorder, an infection, metabolic diseases or combinations thereof.

Joint inflammatory disorders include, but are not limited to inflammatory joint disease, ankylosis, synovitis, arthralgias, spondylitis, bursitis, brucellosis, gout, chondrocalcinosis, degenerative joint disease, arthritis, osteoarthritis, infectious arthritis, Still's disease, onchronotic arhtropathy, mucopolysaccharidoses, osteochondritis dissecans, synovial diabetes, Lyme disease, dysplasia, joint injury, sprains, dislocations, scarring or following joint surgery or combinations thereof.

Joint immune disorders include but are not limited to rheumatism and lupus erythematosus. Joint proliferative disorders include but are not limited to, synovial chondromatosis, osteochondromatosis and synoviomas.

Described herein, in certain embodiments, are injectable formulations for intra-articular or peri-articular administration that include a leukotriene synthesis inhibitor compound, wherein the injectable formulation is administered to treat joint pain or inflammation. In some embodiments, an injectable formulation for intra-articular or peri-articular administration disclosed herein comprises a therapeutically-effective amount of a leukotriene synthesis inhibitor. In some embodiments, an injectable formulation for intra-articular or peri-articular administration disclosed herein is administered before or after joint disorder, disease or condition. In some embodiments, an injectable formulation for intra-articular or peri-articular administration disclosed herein is administered before or after contact with an allergen and/or irritant and/or an infectious agent (e.g., a virus, bacteria or fungus). In some embodiments, an injectable formulation for intra-articular or peri-articular administration disclosed herein is administered before or after a physical trauma (e.g., surgery, or injury). In one aspect, an injectable formulation for intra-articular or peri-articular administration disclosed herein that includes a leukotriene synthesis inhibitor is administered to a joint and/or a tissue related thereto to treat and prevent joint pain following surgery. It is understood that an injectable formulation for intra-articular or peri-articular administration disclosed herein is applied to the site of injury. In some embodiments, an injectable formulation described herein comprises a leukotriene synthesis inhibitor compound in combination with an additional therapeutic agent (e.g. analgesic) and is intra-articularly administered and peri-articular administration to a joint and/or a tissue related thereto to treat and prevent joint pain following surgery.

In certain instances, leukotrienes and other 5-lipoxygenation products are involved in pain sensitization. In one aspect, inhibiting the activity of cPLA₂, FLAP and/or 5-LO inhibits the activity leukotriene-mediated pain sensitization. In some embodiments, an injectable formulation described herein comprises a leukotriene synthesis inhibitor compound in combination with an additional therapeutic agent (e.g., analgesic) and is administered to a joint and/or a related tissue and inhibits the activity of pain sensitization, and/or treats joint pain.

In one aspect, leukotrienes and 5-lipoxygenation products are involved in the pathogenesis of joint pain or inflammation described herein. Inhibition of leukotriene and 5-lipoxygenation product synthesis will result in a decrease in the production of leukotrienes, 5-HETE and 5-oxo-ETE. A reduction of the amount of leukotrienes and 5-lipoxygenation products results in a decrease of the pain symptoms associated with such joint disorders. In one aspect, the reduced amounts of leukotrienes are reduced amount of cysteinyl leukotrienes. In one aspect, the reduced amounts of leukotrienes are reduced amount of LTB₄. In one aspect the reduced amounts of 5-lipoxygenation products are reduced amount of 5-oxo-ETE.

In one aspect, cytokines are involved in the pathogenesis of joint pain described herein. In some instances, inhibition of leukotriene synthesis will result in a decrease in the production of at least one cytokine In one aspect, the cytokine is interleukin 8 (IL-8). A reduction of IL-8 results in a decrease of the pain symptoms associated with such joint disorders. In some embodiments, an injectable formulation described herein comprises a leukotriene synthesis inhibitor compound in combination with an additional therapeutic agent (e.g. analgesic) and is intra-articularly administered to a joint and/or a tissue related and inhibits the production of cytokines.

### FLAP Inhibitors

In one aspect, the leukotriene synthesis inhibitor compound is selected from FLAP inhibitor compounds disclosed herein or known in the art.

In one aspect, the FLAP inhibitor is selected from compounds described in U.S. patent application Ser. No. 11/538,762 (issued as U.S. Pat. No. 7,405,302); U.S. patent application Ser. No. 12/131,828; U.S. patent application Ser. No. 11/553,946 (published as 2007/0105866); U.S. patent application Ser. No. 11/925,841; U.S. patent application Ser. No. 12/089,706; U.S. patent application Ser. No. 12/089,707; U.S. patent application Ser. No. 12/092,570; U.S. patent application Ser. No. 11/744,555 (published as 2007/0219206); U.S. patent application Ser. No. 11/746,010 (published as 2007/0225285); U.S. patent application Ser. No. 11/745,387 (published as 2007/0244128); U.S. patent application Ser. No. 12/257,876; U.S. patent application No. 61/055,887; U.S. patent application No. 61/055,899; International Patent Application no. PCT/US07/86188; WO 07/047207; WO07/0560210; WO07/056220; WO07/056228; WO05/009951; WO06/044602; WO06/098912; WO07/056210; WO07/120574; WO08/030369; International Patent Application no. PCT/US08/62310; International Patent Application no. PCT/US08/062793; International Patent Application no. PCT/US08/62580; International Patent Application no. PCT/US2008/052960; International Patent Application no. PCT/US08/81190; International Patent Application no. PCT/US08/76225; each of which is herein incorporated by reference in its entirety.

In one aspect, the FLAP inhibitor is: MK886 (also known as 3-[3-tert-butylsulfanyl-1-(4-chloro-benzyl)-5-isopropyl-1H-indol-2-yl]-2,2-dimethyl-propionic acid); MK591 (also known as 3-[3-tert-butylsulfanyl-1-(4-chloro-benzyl)-5-(quinolin-2-ylmethoxy)-1H-indol-2-yl]-2,2-dimethyl-propionic acid); DG031 (also known as BAY X1005; cyclopentyl-[4-(quinolin-2-ylmethoxy)-phenyl]-acetic acid); 3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid; 3-[3-tert-Butylsulfanyl-1-[4-(5-methoxy-pyrimidin-2-yl)-benzyl]-5-(5-methyl-pyrazin-2-ylmethoxy)-1H-indol-2-yl]-2,2-dimethyl-propionic acid; 3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid; 3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid; 3-[3-tert-Butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid; 3-[3-tert-Butylsulfanyl-1-[4-(5-fluoro-pyridin-2-yl)-benzyl]-5-(quinolin-2-ylmethoxy)-1H-indol-2-yl]-2,2-dimethyl-propionic acid; 2-[3-tert-Butylsulfanyl-1-[4-(5-methoxy-pyrimidin-2-yl)-benzyl]-5-(5-methyl-pyridin-2-ylmethoxy)-1H-indol-2-ylmethyl]-2-ethyl-butyric acid; 3-[3-tert-Butylsulfanyl-1-[4-(6-methoxy-pyridin-3-yl)-benzyl]-5-(5-methyl-pyridin-2-ylmethoxy)-1H-indol-2-yl]-2,2-dimethyl-propionic acid; 3-[5-((S)-1-Acetyl-pyrrolidin-2-ylmethoxy)-3-tert-butylsulfanyl-1-(4-chloro-benzyl)-1H-indol-2-yl]-2,2-dimethyl-propionic acid; 3-[3-tert-butylsulfanyl-1-[4-(5-fluoro-pyridin-2-yl)-benzyl]-5-(pyridin-2-ylmethoxy)-1H-indol-2-yl]-2,2-dimethyl-propionic acid; 3-{5-((S)-1-Acetyl-2,3-dihydro-1H-indol-2-ylmethoxy)-3-tert-butylsulfanyl-1-[4-(5-ethoxy-pyrimidin-2-yl)-benzyl]-1H-indol-2-yl}-2,2-dimethyl-propionic acid); or a pharmaceutically acceptable salt thereof.

In another aspect, the FLAP inhibitor is selected from MK-0591, MK-886, AM103, AM803, AM679, or AM643, or a pharmaceutically acceptable salt thereof.

In another aspect, the FLAP inhibitor is a compound (or a pharmaceutically acceptable salt thereof) described in U.S. Pat. Nos. 4,929,626; 4,970,215; 5,081,138; 5,204,344; 5,126,354; 5,221,678; 5,229,516; 5,272,145; 5,283,252; 5,292,769; 5,304,563; and 5,597,833, each of which is herein incorporated by reference for the disclosure of such FLAP inhibitors.

### 5-LO Inhibitors

In one aspect, the leukotriene synthesis inhibitor compound is selected from 5-LO inhibitor compounds disclosed herein or known in the art.

In one aspect, the 5-LO inhibitor is a compound (or a pharmaceutically acceptable salt thereof) described in U.S. patent application No. 61/245,198, filed Sep. 23, 2009; U.S. patent application No. 61/245,206, filed Sep. 23, 2009; U.S. patent application No. 61/311,184, filed Mar. 5, 2010; U.S. patent application Ser. No. 11/626,222 (published as 2007/0173508); International Patent Application no. PCT/US10/49954, International Patent Application no. PCT/US10/49967 and International Patent Application no. PCT/US07/01618 (published as WO 07/087250); each of which is herein incorporated by reference in its entirety.

In one aspect, the 5-LO inhibitor is zileuton, VIA 2291, ZD2138, AZD4407, MK0633, or CJ-13,610 (PF-4191834), or a pharmaceutically acceptable salt thereof.

In one aspect, the 5-LO inhibitor is a compound (or a pharmaceutically acceptable salt thereof) described in U.S. Pat. Nos. 6,291,520; 6,262,073; 6,194,585; 6,174,883; 6,166,031; 6,121,323; 6,063,928; 6,028,202; 6,025,384; 5,998,451; 5,925,769; 5,919,809; 5,917,057; 5,883,106; 5,840,749; 5,814,648; 5,811,432; 5,798,383; 5,792,882; 5,753,682; 5,750,544; 5,714,633; 5,696,141; 6,875,785; 6,756,399; 6,753,344; 6,696,477; 6,677,364; 6,653,311; 6,515,014; 6,512,121; 6,479,546; 6,455,541; 6,432,999; 6,432,993; 6,420,392; 6,346,624; 6,344,563; 6,294,574; 5,665,768; 5,665,752; 5,665,749; 5,643,933; 5,580,989; 5,576,338; 5,552,437; 5,552,424; 5,541,218; 5,541,205; 5,534,524; 5,527,827; 5,519,022; 5,521,212; 5,514,703; 5,512,594; 5,504,097; 5,504,095; 5,484,805; 5,482,966; 5,478,843; 5,478,842; 5,478,822; 5,462,953; 5,459,271; 5,459,154; 5,457,125; 5,453,439; 5,447,943; 5,466,043; 5,432,181; 5,428,060; 5,428,048; 5,424,320; 5,420,298; 5,418,231; 5,410,054; 5,407,945; 5,403,859; 5,403,857; 5,401,751; 5,399,577; 5,393,764; 5,391,747; 5,384,320; 5,384,318; 5,376,680; 5,373,007; 5,367,079; 5,364,944; 5,364,877; 5,360,815; 5,359,063; 5,350,761; 5,350,754; 5,350,744; 5,342,838; 5,334,614; 5,334,600; 5,332,757; 5,326,907; 5,321,025; 5,314,900; 5,314,898; 5,314,891; 5,308,852; 5,302,603; 5,302,594; 5,300,655; 5,298,512; 5,298,511; 5,292,900; 5,290,802; 5,288,742; 5,283,361; 5,281,720; 5,280,047; 5,278,177; 5,276,037; 5,272,173; 5,260,451; 5,260,316; 5,260,294; 5,258,399; 5,256,789; 5,254,731; 5,254,581; 5,254,533; 5,252,599; 5,252,562; 5,250,565; 5,250,547; 5,248,685; 5,240,941; 5,236,948; 5,236,919; 5,234,950; 5,229,421; 5,227,399; 5,225,438; 5,221,677; 5,220,059; 5,219,881; 5,217,978; 5,217,977; 5,217,971; 5,217,969; 5,217,968; 5,214,070; 5,214,069; 5,212,189; 5,208,262; 5,208,259; 5,206,255; 5,202,326; 5,202,321; 5,196,419; 5,185,377; 5,179,115; 5,175,183; 5,166,161; 5,162,365; 5,145,858; 5,141,950; 5,140,047; 5,137,913; 5,134,150; 5,134,148; 5,132,328; 5,132,319; 5,132,310; 5,130,485; 5,128,364; 5,126,365; 5,124,342; 5,124,070; 5,120,758; 5,120,752; 5,114,958; 5,112,848; 5,105,020; 5,098,932; 5,098,930; 5,093,356; 5,093,351; 5,086,062; 5,086,052; 5,068,251; 5,066,658; 5,059,609; 5,037,853; 5,032,588; 5,026,729; 5,023,255; 5,015,661; 5,008,390; 5,002,941; 4,992,464; 4,987,231; 4,970,210; 4,966,973; 4,963,576; 4,963,541; 4,939,133; 4,916,226; 4,897,422; 4,877,881; 4,873,259; 4,861,878; 4,861,798; 4,822,811; 4,822,809; 4,803,279; 4,792,560; 4,784,997; 4,769,387; 4,737,519; 4,698,344; 4,694,018; 4,686,231; 4,673,684; 4,602,023; 2005/0203145; 5,356,921; U.S. Pat. Application No. 2005/0059665; 2004/0248943; 2004/0209288; 2004/0147565; 2003/0176708; 2003/0119886; 2003/0073722; 2006/0116406; 2007/0149579; 2008/0188521; and WO 2008/095293; WO 2007/038865; WO 2007/016784; WO 2009/042098; WO 2008/065493; and WO 2009/069044 each of which is herein incorporated by reference for the disclosure of such 5-LO inhibitors.

cPLA₂ Inhibitors

In one aspect, the leukotriene synthesis inhibitor compound is selected from cPLA₂ inhibitor compounds disclosed herein or known in the art.

In one aspect, the cPLA₂ inhibitor is methyl arachidonyl fluorophosphonate (MAFP), pyrroxyphene, ONO-RS-082, 1-[3-(4-octylphenoxy)-2-oxopropyl]indole-5-carboxylic acid, 1-[3-(4-octylphenoxy)-2-oxopropyl]indole-6-carboxylic acid, arachidonyl trifluoromethyl ketone, D609, 4-{3-[5-chloro-2-(2-{[(3,4-dichlorobenzyl)sulfonyl]amino}ethyl)-1-(diphenylmethyl)-1H-indol-3-yl]propyl}benzoic acid (WAY-196025), efipladib, 4-{2-[5-chloro-2-(2-{[(3,4-dichlorobenzyl)sulfonyl]amino}-ethyl)-1-(diphenylmethyl)-1H-indol-3-yl]ethoxy}benzoic acid, Ecopladib, (E)-N-[(2S,4R)-4-[N-(biphenyl-2-ylmethyl)-N-2-methylpropylamino]-1-[2-(2,4-difluorobenzoyl)benzoyl]pyrrolidin-2-yl]methyl-3-[4-(2,4-dioxothiazolidin-5-ylidenemethyl) phenyl]acrylamide (RSC-3388), berberine, or glutamine, or a pharmaceutically acceptable salt thereof.

In some embodiments, pharmaceutically acceptable salts are obtained by reacting a leukotriene synthesis inhibitor compound with acids. Pharmaceutically acceptable salts are also obtained by reacting a leukotriene synthesis inhibitor compound with a base. Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, leukotriene synthesis inhibitor compound described herein are reacted with an organic base, such as, but not limited to, ethanolamine, diethanolamine, triethanolamine, tromethamine, N-methylglucamine, dicyclohexylamine, tris(hydroxymethyl)methylamine. In other cases, leukotriene synthesis inhibitor compound forms a salt with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like. In some embodiments, compounds are prepared as a sodium salt.

In some embodiments, the leukotriene synthesis inhibitor compound is included in the formulations described herein as pharmaceutically acceptable salts, and/or pharmaceutically acceptable solvates. In some embodiments, a leukotriene synthesis inhibitor compound is included in the formulations described herein in free acid form or free base form.

In some embodiments, the leukotriene synthesis inhibitor compound described herein possesses one or more stereocenters and each center exists independently in either the R or S configuration. The compounds presented herein include all diastereomeric, enantiomeric, and epimeric forms as well as the appropriate mixtures thereof.

### Joint Pain and Inflammation

In certain embodiments, the formulation is administered to treat joint pain or inflammation associated an immune disorder (e.g. an autoimmune disorder); a proliferation disorder (e.g., synoviomas); contact with an allergen, and/or an irritant; a fibroblast disorder (e.g., scarring); a physical trauma (e.g. surgery) or combinations thereof. Described herein, in certain embodiments, are injectable formulations for intra-articular or peri-articular administration, wherein the formulation is administered to treat pain associated with inflammatory joint disease, ankylosis, synovitis, arthraglias, rheumatism, spondylitis, bursitis, brucellosis, gout, chondrocalcinosis, degenerative joint disease, lupus erythematosus, arthritis, osteoarthritis, infectious arthritis, Still's disease, onchronotic arrhropathy, mucopolysaccharidoses, osteochondritis dissecans, synovial chondromatosis, synoviomas, diabetes, Lyme disease, dysplasia, joint injury, sprains, dislocations, tears in ligaments and tendons, hemarthrosis, scarring or following joint surgery or combinations thereof. In some embodiments, an injectable formulation for intra-articular or peri-articular administration disclosed herein comprises a therapeutically-effective amount of a leukotriene synthesis inhibitor.

In some embodiments, an injectable formulation for intra-articular or peri-articular administration disclosed herein is administered before or after contact with an allergen and/or irritant and/or an infectious agent (e.g., a virus). In some embodiments, an injectable formulation for intra-articular or peri-articular administration disclosed herein is administered before or after a physical trauma (e.g., surgery, or injury). In some embodiments, the injectable formulations described herein are administered for conditions associated with the articular capsule surrounding the joint. In other embodiments, the injectable formulations are intra-synovially administered to the joint, but subsequently penetrate into the related tissues to treat a disease associated joint pain. In other embodiments, the injectable formulations are administered in the synovial cavity of the joint and related tissues: non-limiting examples include the synovial fluid, synovial membrane, articular cartilage and ligaments.

In some embodiments, an injectable formulation for intra-articular or peri-articular administration disclosed herein is administered in order to reduce the use of systemic opiate therapy after a physical trauma (e.g., surgery, or injury). In some embodiments, the injectable formulations described herein are administered for conditions associated with the articular capsule surrounding the joint. In other embodiments, the injectable formulations are intra-synovially administered to the joint, but subsequently penetrate into the related tissues to treat a disease associated joint pain. In other embodiments, the injectable formulations are administered in the synovial cavity of the joint and related tissues: non-limiting examples include the synovial fluid, synovial membrane, articular cartilage and ligaments.

In some embodiments, an injectable formulation for intra-articular or peri-articular administration disclosed herein is administered to treat fibromylagia pain. In other embodiments, an injectable formulation for intra-articular or peri-articular administration disclosed herein is administered to treat back pain. In other embodiments, an injectable formulation for intra-articular or peri-articular administration disclosed herein is administered to treat allodynia.

### Certain Terminology

The terms “individual,” “patient,” or “subject” are used interchangeably. As used herein, they mean any mammal. In one aspect, the mammal is a human.

The terms “treat,” “treating” or “treatment,” and other grammatical equivalents as used herein, include alleviating, abating, inhibiting, reducing, ameliorating, delaying the onset of, arresting the progression of, and/or inducing the regression of a disorder and/or the symptom(s) of a disorder. The terms also include prophylactic treatment of a disorder. The terms further include achieving any therapeutic benefit. Therapeutic benefit means the eradication or amelioration of the underlying disorder being treated, and/or the eradication or amelioration of one or more of the physiological symptoms associated with the underlying disorder such that an improvement is observed in the individual.

The terms “prevent,” “preventing” or “prevention,” and other grammatical equivalents as used herein include inhibiting (arresting or stopping) the development of a disorder, and/or inhibiting (arresting or stopping) the further progression of a disorder. These terms are intended to include prophylaxis. For prophylactic benefit, a formulation disclosed herein is administered to an individual at risk of developing a particular disorder, or to an individual reporting one or more of the physiological symptoms of a disease, or to an individual at risk of reoccurrence of the disease.

The terms “effective amount” or “therapeutically effective amount” as used herein, refer to an amount of an agent (e.g., a leukotriene synthesis inhibitor compound) being administered which achieve a desired result, e.g., to relieve to some extent one or more symptoms of a disease, disorder or condition being treated. In certain instances, the result is a reduction and/or alleviation of at least one sign, symptom, or cause of a disease, or any other desired alteration of a biological system.

The terms “administer,” “administering,” “administration,” and the like, as used herein, refer to the methods that are used to enable delivery of leukotriene synthesis inhibitors to the desired site of biological action (e.g., the site of pain in a joint). These methods include any suitable method for intra-synovial administration of a leukotriene synthesis inhibitor to a joint. The terms “intra-synovial administration”, “intra-articular administration”, “peri-articular administration” or “intrasynovial administration” and the like, as used herein, refer to the methods that are used to enable delivery of leukotriene synthesis inhibitors to the joint and related tissues including but not limited to, joint spaces, cavities and synovial fluid.

The term “joint” as used herein, refers to any joint in the body and includes without limitation, the hip, knees, shoulders, ankles, elbows, wrists, toes, fingers, and spinal facet joints. Joints in the body include without limitation, ball and socket joints, saddle joints, pivot joints, hinge joints, condyloid joints, and gliding joints. The structures of joints include opposing bones having respective opposing hyaline cartilage articular surfaces; a peripheral, collagenous ligamentous capsule connecting the articular surfaces and defining a central joint space; a synovial membrane lining an inner wall of the capsule, and synovial fluid contained within the joint space.

### Injectable Formulations for Intra-Articular or Peri-Articular Administration

In some embodiments, an injectable formulation for intra-articular or peri-articular administration disclosed herein facilitates the delivery of a leukotriene synthesis inhibitor compound to a joint or a tissue related thereto for a local effect (i.e., an effect that is limited to joint or a tissue related thereto). In certain instances, local administration of a leukotriene synthesis inhibitor compound reduces or eliminates side-effects that are associated with systemic administration of a leukotriene synthesis inhibitor.

In some embodiments, an injectable formulation for intra-articular or peri-articular administration disclosed herein allows compatibility with the viscosity of the synovial fluid. Normal synovial fluid in the joints is highly viscous and functions as a lubricant to reduce friction in the joints. In certain instances, the injectable formulation for intra-articular or peri-articular administration administration is formulated to have a viscosity about the same to the viscosity of the normal synovial fluid. In certain instances, the injectable formulation for intra-articular or peri-articular administration is formulated to not change or modify the viscosity of the normal synovial fluid. In certain instances, the injectable formulation for intra-articular or peri-articular administration is formulated to modify or change the viscosity of diseased synovial fluid to about the same as the viscosity of normal synovial fluid.

In some embodiments, a leukotriene synthesis inhibitor for intra-articular or peri-articular administration is formulated as any injectable form. In some embodiments, a leukotriene synthesis inhibitor for intra-articular or peri-articular administration is formulated as an injectable solution, suspension (e.g., an non-aqueous suspension), emulsion, gel, liposome, niosome, pharmacosome, nanoparticle, powders to be reconstituted as solution or combinations thereof. In some embodiments, a leukotriene synthesis inhibitor for intra-articular or peri-articular administration is administered with additional injection excipients. In some embodiments, a leukotriene synthesis inhibitor for intra-articular or peri-articular administration is administered via devices for injection, infusion, implantation or insertion (e.g., via an biodegradable insert or a soluble insert) or combinations thereof.

In some embodiments, a leukotriene synthesis inhibitor for intra-articular administration is formulated as a controlled release formulation. In some embodiments, a leukotriene synthesis inhibitor for intra-synovial administration is formulated in combination with other therapeutic agents.

### Vehicles

Disclosed herein, in certain embodiments, is an injectable formulation for administration wherein the intra-articular or peri-articular formulation for administration is in the form of an injectable solution, suspension (e.g., an non-aqueous suspension), emulsion, gel, liposome, niosome, pharmacosome, nanoparticle, or powder to be reconstituted as solution. In certain instances, water is used as a vehicle for an injectable formulation. In certain instances, solvents miscible with water are used to increase solubility of a leukotriene synthesis inhibitor. Solvents miscible with water include, but are not limited to, ethyl alcohol, polyethylene glycol, and polypropylene glycol. In certain instances, cyclodextrin and derivatives are used to increase solubility of a leukotriene synthesis inhibitor for intra-articular or peri-articular administration disclosed herein. Cyclodextrin derivatives that can be used as a solubilizing agent include, but are not limited to, α-cyclodextrin, β-cyclodextrin, γ-cyclodextrin, hydroxyethyl β-cyclodextrin, hydroxypropyl γ-cyclodextrin, hydroxypropyl β-cyclodextrin, sulfated β-cyclodextrin, sulfated α-cyclodextrin, sulfobutyl ether β-cyclodextrin.

In other instances, non-aqueous vehicles are used for an injectable formulation. Non-aqueous vehicles include, but are not limited to, fixed oils (e.g., corn oil, cottonseed oil, peanut oil, sesame oil, safflower oil, castor oil, coconut oil, menhaden oil, palm kernel oil, palm oil, soybean oil, rapeseed oil, linseed oil, rice bran oil, pine oil, or sunflower seed oil), or petroleum (e.g., mineral oil, or petroleum jelly).

### Viscosity Enhancers

Disclosed herein, in certain embodiments, is an injectable formulation for intra-articular or peri-articular administration wherein the injectable formulation comprises viscosity enhancers. As used herein, viscosity enhancers refer to agents that increase compatibility and/or iso-viscosity with the synovial fluid; and aid in the formulation of the injectable solution, suspension (e.g., an non-aqueous suspension), emulsion, gel, liposome, niosome, pharmacosome, nanoparticle, or powder to be reconstituted as solution.

Viscosity enhancers include, but are not limited to, hyaluronic acid, modified hyaluronic acid, sodium hyaluronate solutions for treatment of articular disorders such as those sold under the tradename ORTHOVISC (DePuy Ortho Biotech products, L. P., Raritan, N.J.), collagen, poly(alkylene oxide)-based polymers such as polyethylene glycol, chitosan, fucans, copolymers of polysaccharides with degradable polymers, gelatin, starch, cellulose, cellulose derivatives (e.g., regenerated cellulose, methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, lubricin, recombinant lubricin or fragments thereof, LUB:1, and any viscosupplement formulations. In one embodiment, the viscosity enhancers comprise sodium hyaluronate solutions and modified hyaluronic acid. In one embodiment, the viscosity enhancers are in a concentration of 0.1 to 5, 0.5 to 4, 1 to 3, 1.5 to 2.5 percent weight/weight (w/w).

### Tonicity Agents

Disclosed herein, in certain embodiments, is an injectable formulation for intra-articular or peri-articular administration wherein the injectable formulation comprises tonicity agents. Tonicity agents increase isotonic compatibility with physiological environments of the joints and aid in reducing pain and tissue irritation in the injection. Tonicity agents include, but are not limited to electrolytes and saccharides (e.g., dextrose, lactose, mannitol, glucose, dextran, ammonium chloride, sodium chloride, calcium chloride, potassium chloride, sodium bicarbonate, sodium lactate, Ringer's solution, Lactated Ringer's solution, Normosol R, saline, Hartmann's solution). In one embodiment, the tonicity agents comprise Ringer solutions and dextrose. In another embodiment, the tonicity agents comprise an electrolyte mixture of mannitol, sodium chloride, sodium lactate, potassium chloride, and calcium chloride.

### Stability Enhancers:

Disclosed herein, in certain embodiments, is an injectable formulation for intra-articular or peri-articular administration wherein the injectable formulation comprises stability enhancers. Stability enhancers aid in stabilizing the injectable formulation, preventing degradation of the active ingredient and increasing storage-life of the formulation. Stability enhancers include but are not limited to buffers (e.g., citrates, phosphates, acetates and other acid salts, carbonates), antioxidants (e.g., sodium bisulfite, sulfurous acid salts, ascorbic acid, tocopherols, tocotrienols) antimicrobials (e.g., benzyl alcohol, phenol, parapens, cresol, chlorobutanol, benzoates, sulfates, sorbic acid, sorbates), and cryo- or lyoprotectants (sugars such as sucrose or trehalose; amino acids such as glycine or lysine; or polymers such as liquid polyethylene glycol or dextran; polyols such as mannitol or sorbitol). In one instance, the injectable formulation for intra-articular or peri-articular administration comprises one stability enhancer. In another instance, the injectable formulation for intra-articular or peri-articular administration comprises multiple stability enhancers.

### Delivery Devices for Intra-Articular or Peri-Articular Administration

In some embodiments, an injectable formulation for intra-articular or peri-articular administration disclosed herein is administered or delivered via a syringe and needle, where it releases a leukotriene synthesis inhibitor into the joint. In one instance, the formulation is injected through a 16 to 24 gauge needle adjusted for formulation composition and site of administration. The syringe used is manufactured from any known method and includes, without limitations, luer-lock syringes, auto-injector syringes, multi-dose syringes, and the like. In one instance, the injectable formulation is delivered via a one-use syringe and needle pre-filled with a leukotriene synthesis inhibitor formulation. In some embodiments, an injectable formulation for intra-articular or peri-articular administration disclosed herein is administered or delivered via known medical methods, including without limitation, catheters, infusion pumps, pen devices, and the like.

### Controlled Release Formulations

In some embodiments, an injectable formulation for intra-articular or peri-articular administration disclosed herein is administered or delivered via a controlled release formulation. Controlled release formulation, as used herein, refers to a formulation that delivers a drug at a predetermined rate for a specific time period. Controlled release formulations include, but are not limited to, depot preparations. Non-limiting examples of depot preparations include solid matrices, liquid preparations, gel preparations, nanoparticle preparations, liposome preparations, niosome preparations, pharmacosome preparations, and microemulsions.

As used herein, a depot preparation is a controlled-release formulation that is injected, implanted or inserted into a joint cavity or a tissue related thereto (e.g., synovial cavity) that provides slow absorption of the drug from the site of administration. The ratio of leukotriene synthesis inhibitor to controlled-release matrix and the nature of the matrix employed control the rate of drug release. In certain instances, a depot preparation is a solid matrix, wherein the solid matrix includes microencapsulated matrices (also known as microencapsule matrices) of a leukotriene synthesis inhibitor in biocompatible, biodegradable polymers. The solid matrix releases a leukotriene synthesis inhibitor over time as the biocompatible, biodegradable polymers degrade over time.

### Nanoparticle Formulations

In certain instances, a depot preparation is a liquid or a gel that include a leukotriene synthesis inhibitor with biocompatible, biodegradable polymers or excipients. In some embodiments, a liquid or gel depo preparation is formulated by forming particles of a leukotriene synthesis inhibitor encapsulated in biocompatible, biodegradable polymers. The particles are in size ranges sufficiently effective for use in an injectable formulation or for delivery or infusion through a hypodermic needle or catheter. In one instance, the nanoparticle sizes range from 1 to 2000, 10 to 1000, 100 to 800, 200 to 600, or 300 to 400 nanometers. In one instance, the nanoparticle sizes range from 300 to 400 nanometers. In one instance, nanoparticles with ranging from 300 to 400 nanometers are mixed with other range sized nanoparticles. For example, nanoparticles ranging from 300 to 400 nanometers are mixed with nanoparticles with range from 150 to 200 nanometers. In certain instances, the particles have sizes ranging from 1 micron to 500 microns. Several conventional methods have been commonly utilized for making biodegradable polymeric particles. Exemplary methods for making nanoparticles include double emulsion/solvent evaporation, the coacervation process and spray drying.

In some embodiments, the particles of biocompatible, biodegradable polymers encapsulate and coat the leukotriene inhibitor compound in the form of microspheres. In some embodiments, the particles of biocompatible, biodegradable polymers are covalently bound to the leukotriene inhibitor compound.

The biocompatible, biodegradable polymers, as used herein, are made from conventional natural and synthetic materials and by any known method. As used herein, biocompatible material refers to a material that is not toxic to the human body. As used herein, a biodegradable material refers to a material that is degraded by bodily processes (e.g., enzymatic) to products readily disposable by the body or absorbed into body tissue. The biodegraded products should also be biocompatible with the body.

Non-limiting examples of biocompatible, biodegradable polymers that are used to make the biodegradable nanoparticles include poly(alpha-hydroxy acid) polymers such as poly(lactic acid) (PLA), poly(glycolic acid) (PGA, copolymers of lactic acid and glycolic acid (PLGA), polyoxalates, polycaprolactone (PCL), copolymers of caprolactone and lactic acid (PCLA), poly(ether ester) multiblock copolymers based on poly(ethylene glycol) and poly(butylene terephthalate), tyrosine-derived polycarbonates, poly(hydroxybutyrate), polydioxanone, poly(alkylcarbonate), poly(orthoesters), polyesters, poly(hydroxyvaleric acid), poly(malic acid), poly(tartaric acid), poly(acrylamides), polyanhydrides, and polyphosphazenes. Suitable polymeric materials also include waxes such as glycerol mono-and distearate and the blends thereof. In certain instances, the nanoparticles include poly (alpha-hydroxy acid) polymers such as poly(lactic acid) (PLA), poly(glycolic acid (PGA), and copolymers of lactic acid and glycolic acid (PLGA). In one instance, the polymers are endcapped with hydrophobic groups such as lauryl ester.

In some embodiments, a depot preparation is formulated by entrapping a leukotriene synthesis inhibitor in liposomes, niosomes, pharmacosomes or microemulsions. In one instance, the liposomes, niosomes, pharmacosomes or microemulsions comprise lipids, phospholipids, cholesterol and other biomacromolecules.

An injectable formulation for intra-articular or peri-articular administration disclosed herein is formulated in any suitable manner. Any suitable technique, carrier, and/or excipient is contemplated for use with the leukotriene synthesis inhibitors disclosed herein. For a summary of intra-articular or peri-articular formulations for administration described herein see Remington: The Science and Practice of Pharmacy, Nineteenth Ed. (Easton, Pa.: Mack Publishing Company, 1995); Hoover, John E., Remington's Pharmaceutical Sciences, Mack Publishing Co., Easton, Pa. 1975; Liberman, H.A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), which are herein incorporated by reference for such disclosures.

### Dosing

Disclosed herein, in certain embodiments, is an injectable formulation for intra-articular or peri-articular administration, wherein the injectable formulation for intra-articular or peri-articular administration is administered for prophylactic and/or therapeutic treatments. In certain instances, amounts effective for this use will depend on the severity and course of the disease, disorder or condition, previous therapy, the individual's health status and response to the drugs, the site of administration, and the judgment of the treating physician. In some embodiments, the dose is about 0.001% by weight to about 10% by weight.

The compounds described herein are administered to any joint in the body, generally referred to as “intra-articular administration,” “peri-articular administration,” “intra-synovial administration” or “intrasynovial administration.” Intra-synovial administration encompasses, but is not limited to administration to the intra-synovial space in the knee, shoulder, temporo-mandibular and corpo-metacarpal joints, elbow, hip, wrist, finger, toes, ankle, and lumbar zygaopophysial (facet) joints in the spine.

Administration of an injectable formulation for intra-articular or peri-articular administration disclosed herein generally results in direct contact of a leukotriene synthesis inhibitor with the synovial fluid. In certain instances, an injectable formulation for intra-articular or peri-articular administration disclosed herein has an effective residence time in joint of about 2 hours, or about 4 hours, or 6 about hours, or 12 about hours, or 18 about hours, or 1 day, or 2 days, or 3 days, or 4 days, or 5 days, or 6 days, or 7 days, or 10 days, or 12 days, or 14 days, or 18 days, or 21 days, or 25 days, or 30 days, or 45 days, or 2 months or 3 months or 4 months or 5 months or 6 months or 9 months or 1 year.

Useful injectable formulations for intra-articular or peri-articular administration can be a solution, suspension, solution/suspension, emulsion, gel, liposome, niosome, pharmacosome, nanoparticle, powders to be reconstituted as solution or combinations thereof. A desired dosage can be administered via a set number of injections or the amount per injection. For example, for a syringe volume of 0.5 ml, administration of 1-6 injections will deliver 0.5-3 ml of an injectable formulation for intra-articular or peri-articular administration disclosed herein. Injectable formulations typically contain from about 0.01% to about 50%, more typically about 0.1% to about 20%, still more typically about 0.2% to about 10%, and most typically about 0.5% to about 5%, weight/volume of a leukotriene synthesis inhibitor.

In some embodiments, where joint pain does not improve, an injectable formulation for intra-articular or peri-articular administration disclosed herein is administered chronically (i.e., for an extended period of time, including throughout the duration of the individual's life). In some embodiments, where a joint pain does improve, an injectable formulation for intra-articular or peri-articular administration disclosed herein is given continuously; alternatively, the dose of leukotriene synthesis inhibitor being administered is temporarily reduced or temporarily suspended for a certain length of time (i.e., a “drug holiday”). In some embodiments, a drug holiday lasts between 2 days and 1 year, including all integers in between. In some embodiments, the dose reduction during a drug holiday is from about 10% to about 100%, including all integers in between.

In some embodiments, where a joint pain does improve, an injectable formulation for intra-articular or peri-articular administration disclosed herein is administered as a maintenance dose. In some embodiments, where a joint pain does improve, an injectable formulation for intra-articular or peri-articular administration disclosed herein is administered with reduced frequency or at a reduced dose.

In one embodiment, an injectable formulation for intra-articular or peri-articular administration disclosed herein is formulated for immediate release of a leukotriene synthesis inhibitor. In some embodiments, a leukotriene synthesis inhibitor is released immediately, or within 1 minute, or within 5 minutes, or within 10 minutes, or within 15 minutes, or within 30 minutes, or within 60 minutes or within 90 minutes.

In one embodiment, an injectable formulation for intra-articular or peri-articular administration disclosed herein is formulated for delayed (or controlled) release of a leukotriene synthesis inhibitor. In some embodiments, a leukotriene synthesis inhibitor compound is released over a time period exceeding 15 minutes, or 30 minutes, or 1 hour, or 4 hours, or 6 hours, or 12 hours, or 18 hours, or 1 day, or 2 days, or 3 days, or 4 days, or 5 days, or 6 days, or 7 days, or 10 days, or 12 days, or 14 days, or 18 days, or 21 days, or 25 days, or 30 days, or 45 days, or 2 months or 3 months or 4 months or 5 months or 6 months or 9 months or 1 year.

In some embodiments, an injectable formulation for intra-articular or peri-articular administration disclosed herein is formulated for immediate and delayed (or controlled) release of a leukotriene synthesis inhibitor.

### Combination Therapy

In one aspect, pharmaceutical compositions and methods disclosed herein include an additional therapeutic agent. In one aspect, the additional therapeutic agent is another leukotriene synthesis inhibitor compound. In one aspect, the additional therapeutic agent is a therapeutic agent other than a leukotriene synthesis inhibitor compound.

In one aspect, the injectable formulations disclosed herein that include a leukotriene synthesis inhibitor compound are co-administered with (either separately or in the same formulation) a therapeutic agent other than a leukotriene synthesis inhibitor compound.

In one aspect, the injectable formulations disclosed herein that include a leukotriene synthesis inhibitor compound are co-administered with (either separately or in the same formulation) a therapeutic agent selected from: antibiotics (e.g., polymyxin B sulfate/bacitracin zinc, polymyxin B/neomycin/gramicidin, polymyxin B/trimethoprim, polymyxin B/bacitracin, fluoroquinolones (e.g., ciprofloxacin, moxifloxacin, ofloxacin, gatifloxacin, levofloxacin), aminoglycosides (e.g. tobramycin, azithromycin, gentamicin, erythromycin, bacitracin); anti-Fungal Agents (e.g., amphotericin B, intraconazole, fluconazole, voriconazole); steroid anti-inflammatory agents (e.g., fluorometholone acetate, prednisolone acetate, loteprednol etabonate, prednisolone sodium phosphate, prednisolone sodium, rimexolone, fluorometholone acetate, dexamethasone, budesonide); non-steroidal anti-inflammatory agents (e.g., nepafenac, ketorolac tromethamine, bromfenac, diclofenac sodium, ketorolac tromethamine, ketotifen fumarate); antihistamines (e.g., emedastine difumarate, olopatadine hydrochloride, epinastine HCl, Azelastine Hydrochloride, ketotifen fumarate); antivirals (e.g., acyclovir, vidarabine, trifluridine); alpha agonists (e.g., apraclonidine, brimonidine, bimatoprost); beta blockers (e.g., betaxolol hydrochloride, levobunolol hydrochloride, carteolol hydrochloride, metipranolol, timolol maleate, timolol hemihydrate); carbonic anhydrase inhibitors (e.g., brinzolamide, dorzolamide, acetazolamide); miotics (e.g., acetylcholine chloride, echothiophate); prostaglandins (e.g., travoprost, bimatoprost, latanoprost); anti-angiogenesis agents (e.g., pegaptanib sodium, ranibizumab, verteporfin); mast cell stabilizers (e.g., lodoxamide tromethamine, nedocromil sodium, cromolyn sodium, pemirolast potassium), opiod analgesics (e.g., morphine, hydromorphone, oxymorphone, codeine, oxycodone, tramadol, fentanyl), anesthetics (e.g., lidocaine, bupivacaine, benzocaine, mepivacaine, procaine), and lubricating agents (e.g., lubricin, recombinant lubricin and or fragments thereof, LUB:1, chondroitin, glucosamine, hyaluron, heparan).

In one aspect, the injectable formulations disclosed herein that include a leukotriene synthesis inhibitor compound are co-administered with (either separately or in the same formulation) a therapeutic agent selected from: acetaminophen, non-steroidal anti-inflammatory drugs, COX-2 selective inhibitors, corticosteroids, narcotics, non-narcotic analgesics, anesthetics, capsaicin, hyaluronic acid, glucosamine, chondroitin, S-adenosyl methionine (SAMe), anti-TNF alpha agents.

Anesthetics include, but are not limited to: procaine, benzocaine, chloroprocaine, cyclomethycaine, dimethocaine, larocaine, propoxycaine, procaine, novocaine, proparacaine, tetracaine, amethocaine, articaine, bupivacaine, carticaine, cinchocaine, dibucaine, etidocaine, levobupivacaine, lidocaine, lignocaine, mepivacaine, piperocaine, prilocaine, ropivacaine, trimecaine.

NSAIDs include, but are not limited to: aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, flurobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, COX-2 specific inhibitors (such as, but not limited to, celecoxib, rofecoxib, valdecoxib, parecoxib, etoricoxib, lumiracoxib, CS-502, JTE-522, L-745,337 and NS398).

Corticosteroids, include, but are not limited to: betamethasone (celestone), prednisone (deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, rimexolone, tixocortol, triamcinolone, and ulobetasol.

Anti-TNF agents include, but are not limited to: infliximab, adalimumab, certolizumab pegol, golimumab, etanercept.

In one aspect, the injectable formulations disclosed herein that include a leukotriene synthesis inhibitor compound are co-administered with (either separately or in the same formulation) an analgesic and/or and anti-inflammatory compound. Analgesic and/or and anti-inflammatory compounds include, but are not limited to: aspirin, carbaspirin, choline salicylate, diflunisal, magnesium salicylate, salicylamide, salicylic acid, salsalate, sodium thiosalicylate, acetaminophen, phenacetin, aminopyrine, mefenamic acid, methotrimeprazine, oxyphenbutazone, phenylbutazone, indomethacin, ibuprofen, sulindac, piroxicam, meclofenamate, zomepirac, codeine, morphine, meperidine, pethinine, alphaprodine, fentanyl, levorphanol, methadone, phenazocine, butorphanol, ethobeptozine, nalbuphine, pentazocine, propoxyphene, fenoprofen, naproxen, tolmeton and the like.

In some embodiments, the leukotriene synthesis inhibitor and the additional therapeutic agent are in the same pharmaceutical composition. In some embodiments, the leukotriene synthesis inhibitor and the additional therapeutic agent are in separate pharmaceutical compositions. By way of a non-limiting example, a combination therapy includes an injectable formulation of a leukotriene synthesis inhibitor for intra-articular or peri-articular administration with an oral formulation of an analgesic. By way of a non-limiting example, a combination therapy includes an injectable formulation of a leukotriene synthesis inhibitor for intra-articular or peri-articular administration with an oral solid formulation of an opioid analgesic.

In some embodiments, the leukotriene synthesis inhibitor and the additional therapeutic agent are administered at the same time. In some embodiments, the leukotriene synthesis inhibitor and the additional therapeutic agent are administered at different times.

### EXAMPLES

### Example 1

### Formulation of a Leukotriene Synthesis Inhibitor

In one aspect, aqueous injectable solution that includes a leukotriene synthesis inhibitor (e.g. a FLAP inhibitor, 5-LO inhibitor or a c-PLA₂ inhibitor) is prepared by dissolving a leukotriene inhibitor compound in 10% beta-hydroxypropyl cyclodextrin (BHPC) in water.

### Example 2

### Solution of Leukotriene Synthesis Inhibitor

In one aspect, a leukotriene synthesis inhibitor (e.g. a FLAP inhibitor, 5-LO inhibitor or a c-PLA₂ inhibitor) is formulated for injection as follows:

| | |
|---|---|
| leukotriene synthesis inhibitor | 1.0 g |
| Ethyl alcohol | 0.5 mL |
| Ascorbic acid | 0.5 g |
| Chlorobutanol | 0.1 mL |
| Sodium hyaluronate | 10 g |
| Sodium chloride (0.9%) | qs to 100 mL |

### Example 3

### Solution of Leukotriene Synthesis Inhibitor

In one aspect, a leukotriene synthesis inhibitor (e.g. a FLAP inhibitor, 5-LO inhibitor or a c-PLA₂ inhibitor) is formulated for injection as follows:

| | |
|---|---|
| leukotriene synthesis inhibitor | 2.0 g |
| Poly(lactic acid)(PLA) | 1.0 g |
| Sodium citrate | 1 mL |
| Ringer's Solution | qs to 100 mL |

### Example 4

### Solution of Leukotriene Synthesis Inhibitor

In one aspect, a leukotriene synthesis inhibitor (e.g. a FLAP inhibitor, 5-LO inhibitor or a c-PLA₂ inhibitor) is formulated for injection as follows:

| | |
|---|---|
| leukotriene synthesis inhibitor | 1.5 g |
| Copolymers of lactic and glycolic acid (PLGA) | 1.5 g |
| Benzyl Alcohol | qs to 2% |
| Vitamin E | 1 mL |
| Corn Oil | qs to 100 mL |

### Example 5

### Solution of 5-LO Inhibitor

In one aspect, a 5-LO inhibitor is formulated for injection as follows:

| | |
|---|---|
| 5-LO inhibitor | 2.5 g |
| Copolymers of lactic and glycolic acid (PLGA) | 1.5 g |
| Benzyl Alcohol | qs to 2% |
| Vitamin E | 1 mL |
| Corn Oil | qs to 100 mL |

### Example 6

### Clinical Trial Evaluating Effect of Intra-Articular or Peri-Articular Administration of a Leukotriene Inhibitor Compound in Reducing Joint Pain from Osteoarthritis

A single-center, double-blind, randomized, two way cross-over, placebo-controlled study to evaluate the safety and efficacy of intra-articular or peri-articular administration of a leukotriene inhibitor compound to ambulatory individuals with symptomatic tibio-femoral osteoarthritis (OA) (global OA pain in the study knee in the range of 50-80 on a 100 mm VAS score). Other criteria for inclusion are: age of 40 years or older; Kellgren-Lawrence grade II-III by X-ray within 3 last months; absence of tense effusion, mechanical deficit, or recent (<2 years) trauma. Each subject will receive the active treatment (e.g., an injectable formulation of a leukotriene inhibitor compound intra-articularly or peri-articularly administered to the knee) or placebo. 120 patients receiving the active treatment are randomized in six groups:

Group 1—one injection of 6 ml of a leukotriene inhibitor compound;

Group 2—one injection of 4 ml of a leukotriene inhibitor compound;

Group 3—two injections of 4 ml of a leukotriene inhibitor compound two weeks apart;

Group 4—three injections of 4 ml of a leukotriene inhibitor compound four weeks apart;

Group 5—three injections of 2 ml of a leukotriene inhibitor compound four weeks apart;

Group 6—three injections of 4 ml of a leukotriene inhibitor compound 12 weeks apart.

120 patients receiving placebos are randomized in six groups that correspond to groups 1-6.

The patients were then followed up to 6 months (at weeks 2, 3, 8, 16, and 24). The primary and secondary assessment endpoints used are described below.

### A. Patient Self-Assessment of OA Pain

The primary efficacy endpoint of this study is to evaluate the efficacy of an injectable formulation of a leukotriene inhibitor compound in patients with OA of the knee with respect to study knee OA pain relief. This is measured on a patient self assessed 100-mm VAS, with endpoints of no pain (0 mm) to extreme pain (100 mm) within the past 48 hours; performed at 24 weeks following the first injection.

### B. Patient Global Self-Assessment

The patient rates the overall status of their target knee on the 100-mm VAS which ranged from very good (0 mm) to very poor (100 mm), taking into account all related signs and symptoms over the previous 48 hours. The exact instructions presented to the patient are the following: “Please indicate by using a vertical line below, the overall general condition of your (study) knee at the time of this visit. The left or ‘0’ score indicates ‘Very good’ while the ‘100’ score indicates ‘Very poor’.”

### C. WOMAC™

The patient is directed to complete the VAS version of the WOMAC™ as described in Bellamy et al. (1988) J. Rheumatol., 15(12):1833-40. This scale is a tri-dimensional, disease-specific, self-administered, health status measure. It probes clinically important, patient-relevant symptoms in the areas of pain, stiffness and physical function in a total of 24 questions. The WOMAC™ is provided to the patient in the local language and typically is completed in less than 5 minutes. The WOMAC™ sub-sections are the following.

The WOMAC™ Section A consists of questions regarding levels of pain during activity and the responses are scored (by the patient) with a VAS that ranges from no pain (0 mm) to extreme pain (100 mm). Assessment of pain is made for the following scenarios:

| | | |
|---|---|---|
| 1. | Walking on a flat surface? | [walking] |
| 2. | Going up or down stairs? | [stair climbing] |
| 3. | At night while lying in bed? | [nocturnal] |
| 4. | Sitting or lying? | [rest] |
| 5. | Standing upright? | [weight bearing] |

The WOMAC™ Part B (Stiffness Score) consists of questions regarding stiffness severity during activity and the responses are scored (by the patient) with a VAS that ranges from no stiffness (0 mm) to extreme stiffness (100 mm). Assessment of stiffness was made for the following scenarios:

| | | |
|---|---|---|
| 1. | After waking in the morning? | [morning stiffness] |
| 2. | During the rest of the day? | [stiffness occurring later in the day] |

The WOMAC™ Section C consists of questions regarding functional impairment during activity and the responses are scored (by the patient) with a VAS that ranges from no difficulty (Q mm) to extreme difficulty (100 mm). Assessment of functional impairment was made for the following scenarios:

| | | |
|---|---|---|
| 1. | Descending stairs? | [morning stiffness] |
| 2. | Ascending stairs? | [stiffness occurring later in the day] |
| 3. | Rising from sitting? | [rising sitting] |
| 4. | Standing? | [standing] |
| 5. | Bending to the floor? | [bending] |
| 6. | Walking on flat surfaces? | [flat walking] |
| 7. | Getting in and out of car? | [car] |
| 8. | Going shopping? | [shopping] |
| 9. | Putting on socks/stockings | [socks/stockings off] |
| 10. | Lying in bed | [lying in bed] |
| 11. | Getting in/out of bath | [in/out bath] |
| 12. | Sitting | [sitting] |
| 13. | Getting on/off toilet | [on/off toilet] |
| 14. | Heavy domestic duties | [heavy domestic] |
| 15. | Light domestic duties | [light domestic] |

The change from baseline in the total WOMAC™ score derived from the 3 WOMAC™ sections (A, B and C) at all time points following the first injection is analyzed as a secondary endpoint.

### D. Physician OA Global Assessment

After the patient has completed the global assessments and the WOMAC™, the Investigator will rate the overall condition of the patient's knee at the time of the visit on the 100-mm VAS ranging from very good (0 mm) to very poor (100 mm). This evaluation is based on the patient's signs of disease, functional capacity and physical examination. The physician is instructed to indicate the overall general condition of the patient's knee at the time of this visit, using a line presented with the left (“0”) extreme of the line indicating “very good” and the right extreme (“100”) indicating “very poor.”

### Example 7

### Rat Model of Joint Pain

A rat model of joint pain is used to test the effect of intra-articular or peri-articular administration of a leukotriene inhibitor compound to a joint for the treatment of pain. The rat knee-joint pain model is adapted from Tonussi and Ferrera, Pain, (1992) 48(3):421-27 and is designed to study articular incapacitation (AI), defined as the inability of a rat with an experimentally induced arthritis to walk normally. Briefly, Male wistar rats (180-220 g) receive a standard intra-articular or peri-articular injection of carrageenin (1 mg in 50 μl total volume), dissolved in sterile saline, into their right knee joints. After carrageenin injection, animals are put to walk on a steel rotary drum (30 cm wide×50 cm diameter), covered with a fine-mesh non-oxidizable wire screen, which rotates at 3 r.p.m. Specially designed metal gaiters wrap around both hind paws. After placement of the gaiters, the animals are allowed to walk freely to accustom themselves to the gaiters. The right paw is connected via a simple circuit to a microcomputer data input/output port. The paw elevation time (PET) is the time that during a 60 s period the inflamed hind paw is not in contact with the cylinder. This is directly proportional to the articular incapacitation. Increase in PET is assumed to reflect articular inflammatory pain and is due to stimulation of periarticular nociceptors

### Prophylactic Evaluation

To analyze the prophylactic effect of injectable formulations of intra-articular or peri-articular administration of a leukotriene inhibitor, groups of rats receive intra-articular or peri-articular administration of a leukotriene inhibitor in the right knee joint 5 hours to 30 minutes at different doses prior to the articular incapacitation. Control groups receive recive 0.9% w/v sterile saline. The rats are then subjected to articular incapacitation as described above. Assessment of prophylactic effects is measured by changes with the PET. A decrease in PET correlates with a reduction in pain.

### Treatment Evaluation

To analyze the therapeutic effect of injectable formulations of intra-articular or peri-articular administration of a leukotriene inhibitor, groups of rats receive intra-articular or peri-articular administration of a leukotriene inhibitor in the right knee joint 5 minutes to 1 hour at different doses after the articular incapacitation as described above. Control groups receive recive 0.9% w/v sterile saline. Assessment of therapeutic effects is measured by changes with the PET. A decrease in PET correlates with a reduction in pain.

The examples and embodiments described herein are illustrative and various modifications or changes suggested to persons skilled in the art are to be included within this disclosure. As will be appreciated by those skilled in the art, the specific components listed in the above examples may be replaced with other functionally equivalent components, e.g., pharmaceutically acceptable excipients, carriers, diluents, and the like.

1. An injectable formulation comprising a leukotriene synthesis inhibitor compound in an amount effective for the treatment of joint pain or inflammation in a mammal, and at least one suitable pharmaceutically acceptable excipient for intra-articular or peri-articular administration.

2. The injectable formulation of claim 1, wherein the injectable formulation inhibits leukotriene synthesis in a joint of a mammal or antagonizes a leukotriene receptor in a joint of a mammal.

3. The injectable formulation of claim 1, wherein the leukotriene synthesis inhibitor is an inhibitor of 5-lipoxygenase activating protein (FLAP).

4. The injectable formulation of claim 3, wherein the leukotriene synthesis inhibitor is MK886 (3-[3-tert-butylsulfanyl-1-(4-chloro-benzyl)-5-isopropyl-1H-indol-2-yl]-2,2-dimethyl-propionic acid); MK591 (3-[3-tert-butylsulfanyl-1-(4-chloro-benzyl)-5-(quinolin-2-ylmethoxy)-1H-indol-2-yl]-2,2-dimethyl-propionic acid); DG031 (BAY X1005; cyclopentyl[4-(quinolin-2-ylmethoxy)-phenyl]-acetic acid); 3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid; 3-[3-tert-Butylsulfanyl-1-[4-(5-methoxy-pyrimidin-2-yl)-benzyl]-5-(5-methyl-pyrazin-2-ylmethoxy)-1H-indol-2-yl]-2,2-dimethyl-propionic acid; 3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid; 3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid; 3-[3-tert-Butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid; 3-[3-tert-Butylsulfanyl-1-[4-(5-fluoro-pyridin-2-yl)-benzyl]-5-(quinolin-2-ylmethoxy)-1H-indol-2-yl]-2,2-dimethyl-propionic acid; 2-[3-tert-Butylsulfanyl-1-[4-(5-methoxy-pyrimidin-2-yl)-benzyl]-5-(5-methyl-pyridin-2-ylmethoxy)-1H-indol-2-ylmethyl]-2-ethyl-butyric acid; 3-[3-tert-Butylsulfanyl-1-1[4-(6-methoxy-pyridin-3-yl)-benzyl]-5-(5-methyl-pyridin-2-ylmethoxy)-1H-indol-2-yl]-2,2-dimethyl-propionic acid; 3-[5-((S)-1-Acetyl-pyrrolidin-2-ylmethoxy)-3-tert-butylsulfanyl-1-(4-chloro-benzyl)-1H-indol-2-yl]-2,2-dimethyl-propionic acid; 3-[3-tert-butylsulfanyl-1-[4-(5-fluoro-pyridin-2-yl)-benzyl]-5-(pyridin-2-ylmethoxy)-1H-indol-2-yl]-2,2-dimethyl-propionic acid; 3-{5-((S)-1-Acetyl-2,3-dihydro-1H-indol-2-ylmethoxy)-3-tert-butylsulfanyl-1-[4-(5-cthoxy-pyrimidin-2-yl)-benzyl]-1H-indol-2-yl}-2,2-dimethyl-propionic acid); or a pharmaceutically acceptable salt thereof.

5. The injectable formulation of claim 1, wherein the leukotriene synthesis inhibitor is an inhibitor of 5-lipoxygenase (5-LO).

6. The injectable formulation of claim 5, wherein the leukotriene synthesis inhibitor is zileuton, VIA 2291, ZD2138, AZD4407, MK0633, CJ-13,610 (PF-4191834), or a pharmaceutically acceptable salt thereof.

7. The injectable formulation of claim 1, wherein the leukotriene synthesis inhibitor is an inhibitor of cytosolic PLA2 (c-PLA₂).

8. The injectable formulation of claim 7, wherein the leukotriene synthesis inhibitor is methyl arachidonyl fluorophosphonate (MAFP), pyrroxyphene, ONO-RS-082, 1-[3-(4-octylphenoxy)-2-oxopropyl]indole-5-carboxylic acid, 1-[3-(4-octylphenoxy)-2-oxopropyl]indole-6-carboxylic acid, arachidonyl trifluoromethyl ketone, D609, 4-{3-[5-chloro-2-(2-{([(3,4-dichlorobenzyl)sulfonyl]amino}ethyl)-1-(diphenylmethyl)-1H-indol-3-yl]propyl}benzoic acid (WAY-196025), efipladib, 4-{2-[5-chloro-2-(2-{[(3,4-dichlorobenzyl)sulfonyl]amino}-ethyl)-1-(diphenylmethyl)-1H-indol-3-yl]ethoxy}benzoic acid, Ecopladib, (E)-N-[(2S,4R)-4-[N-(biphenyl-2-ylmethyl)-N-2-methylpropylamino]-1-[2-(2,4-difluorobenzoyl)benzoyl]pyrrolidin-2-yl]methyl-3-[4-(2,4-dioxothiazolidin-5-ylidenemethyl) phenyl]acrylamide (RSC-3388), berberine, glutamine, or a pharmaceutically acceptable salt thereof.

9. The injectable formulation of claim 1, further comprising a therapeutically-effective amount of an second compound selected from antibiotics; anti-fungal agent; steroid anti-inflammatory agent; non-steroidal anti-inflammatory agent; antihistamine; antiviral; alpha agonist; beta blocker; carbonic anhydrase inhibitor; prostaglandin; anti-angiogenesis agent; mast cell stabilizer; glycosaminoglycan; anti-cancer agent; analgesics, lubricating agents, p38 kinase inhibitors, chemokine inhibitors, anti-TNF alpha agents, analgesics, anesthetics, glucosamine.

10. A method of treating joint pain in a mammal comprising intra-articularly or peri-articularly administering an injectable formulation comprising a therapeutically-effective amount of a leukotriene synthesis inhibitor compound to at least one joint of a mammal with a joint disease, disorder or condition.

11. The method of claim 10, wherein the injectable formulation inhibits leukotriene synthesis in a joint of a mammal.

12. The method of claim 10, wherein the injectable formulation reduces the amount of or inhibits pain in a joint of a mammal.

13. The method of claim 10. wherein the leukotriene synthesis inhibitor is an inhibitor of 5-lipoxygenase activating protein (FLAP).

14. The method of claim 13, wherein the leukotriene synthesis inhibitor is MK886 (3-[3-tert-butylsulfanyl-1-(4-chloro-benzyl)-5-isopropyl-1H-indol-2-yl]-2,2-dimethyl-propionic acid); MK591 (3-[3-tert-butylsulfanyl-1-(4-chloro-benzyl)-5-(quinolin-2-ylmethoxy)-1H-indol-2-yl]-2,2-dimethyl-propionic acid); DG031 (BAY X1005; cyclopentyl-[4-(quinolin-2-ylmethoxy)-phenyl]-acetic acid); 3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid; 3-[3-tert-Butylsulfanyl-1-[4-(5-methoxy-pyrimidin-2-yl)-benzyl]-5-(5-methyl-pyrazin-2-yltnethoxy)-1H-indol-2-yl]-2,2-dimethyl-propionic acid; 3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid; 3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid; 3-[3-tert-Butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid; 3-[3-tert-Butylsulfanyl-1-[4-(5-fluoro-pyridin-2-yl)-benzyl]-5-(quinolin-2-ylmethoxy)-1H-indol-2-yl]-2,2-dimethyl-propionic acid; 2-[3-tert-Butylsulfanyl-1-[4-(5-methoxy-pyrimidin-2-yl)-benzyl]-5-(5-methyl-pyridin-2-ylmethoxy)-1H-indol-2-ylmethyl]-2-ethyl-butyric acid; 3-[3-tert-Butylsulfanyl-1-[4-(6-methoxy-pyridin-3-yl)-benzyl]-5-(5-methyl-pyridin-2-ylmethoxy)-1H-indol-2-yl]-2,2-dimethyl-propionic acid; 3-[5-((S)-1-Acetyl-pyrrolidin-2-ylmethoxy)-3-tert-butylsulfanyl-1-(4-chloro-benzyl)-1H-indol-2-yl]-2,2-dimethyl-propionic acid; 3-[3-tert-butylsulfanyl-1-[4-(5-fluoro-pyridin-2-yl)-benzyl]-5-(pyridin-2-ylmethoxy)-1H-indol-2-yl]-2,2-dimethyl-propionic acid; 3-{5-((S)-1-Acetyl-2,3-dihydro-1H-indol-2-ylmethoxy)-3-tert-butylsulfanyl-1-[4-(5-ethoxy-pyrimidin-2-yl)-benzyl]-1H-indol-2-yl}-2,2-dimethyl-propionic acid), or a pharmaceutically acceptable salt thereof.

15. The method of claim 10, wherein the leukotriene synthesis inhibitor is an inhibitor of 5-lipoxygenase (5-LO).

16. The method of claim 15, wherein the leukotriene synthesis inhibitor is zileuton, VIA 2291, ZD2138, AZD4407, MK0633, CJ-13,610 (PF-4191834), or a pharmaceutically acceptable salt thereof.

17. The method of claim 10, wherein the leukotriene synthesis inhibitor is an inhibitor of cytosolic PLA₂ (c-PLA₂).

18. The method of claim 17, wherein the leukotriene synthesis inhibitor is methyl arachidonyl fluorophosphonate (MAFP), pyrroxyphene, ONO-RS-082, 1-[3-(4-octylphenoxy)-2-oxopropyl]indole-5-carboxylic acid, 1-[3-(4-octylphenoxy)-2-oxopropyl]indole-6-carboxylic acid, arachidonyl trifluoromethyl ketone, D609, 4-{3[5-chloro-2-(2-{[(3,4-dichlorobenzyl)sulfonyl]amino}ethyl)-1-(diphenylmethyl)-1H-indol-3-yl]propyl}benzoic acid (WAY-196025), efipladib, 4-{2-[5-chloro-2-(2-{[(3,4-dichlorobenzyl)sulfonyl]amino}-ethyl)-1-(diphenylmethyl)-1H-indol-3-yl]ethoxy}benzoic acid, Ecopladib, (E)-N-[(2S,4R)-4-[4N-(biphenyl-2-ylmethyl)-N-2-methylpropylamino]-1-[2-(2,4-difluorobenzoyl)benzoyl]pyrrolidin-2-yl]methyl-3[4-(2,4-dioxothiazolidin-5-ylidenemethyl)phenyl]acrylamide (RSC-3388), berberine, glutamine, or a pharmaceutically acceptable salt thereof.

19. The method of claim 10, further comprising administering to the mammal a therapeutically-effective amount of a compound selected from antibiotics; anti-fungal agent; steroid anti-inflammatory agent; non-steroidal anti-inflammatory agent; antihistamine: antiviral: alpha agonist; beta blocker; carbonic anhydrase inhibitor; prostaglandin; anti-angiogenesis agent; mast cell stabilizer; glycosaminoglycan; anti-cancer agent; analgesics, lubricating agents, p38 kinase inhibitors, chemokine inhibitors, anti-TNF alpha agents, analgesics, anesthetics, glucosamine.
